(19) United States
(12) Patent Application Publication
(10) Pub. No.: US 2026/0088137 A1
(43) Pub. Date: Mar. 26, 2026

(54) SYSTEM FOR PREDICTING PHYSICAL PROPERTIES AND METHOD THEREFOR

(71) Applicant: LG MANAGEMENT DEVELOPMENT INSTITUTE CO., LTD., Seoul (KR)

(72) Inventors: Chang Young PARK, Seoul (KR); Hong Jun Yang, Seoul (KR); Jae Wan Lee, Seoul (KR); Se Hui Han, Seoul (KR)

(21) Appl. No.: 19/409,916

(22) Filed: Dec. 5, 2025

Related U.S. Application Data
(63) Continuation of application No. PCT/KR2025/013535, filed on Sep. 3, 2025.

(30) Foreign Application Priority Data
Sep. 5, 2024 (KR) 10-2024-0120761
Dec. 10, 2024 (KR) 10-2024-0182522
Sep. 1, 2025 (KR) 10-2025-0123021

(51) Int. Cl. G16C 20/30; G06N 3/042; G06N 3/045; G16C 20/70

(52) U.S. Cl. CPC G16C 20/30; G06N 3/042; G06N 3/045; G16C 20/70

(57) ABSTRACT

A system for predicting physical properties of a material which: extracts a “graph embedding” by inputting material information into a first artificial intelligence (AI) model; extracts a “text embedding” by inputting a textual description of a crystal structure of the material into a second AI model; divides the “text embedding” into a plurality of “structural information embeddings”; and combines the “graph embedding” with at least one of the plurality of “structural information embeddings”. The “structural information embeddings” may be categorized into global information, semi-global information, and local information of the crystal structure.

### CROSS REFERENCE TO RELATED APPLICATIONS

This application is a Bypass Continuation of International Patent Application No. PCT/KR2025/013535, filed on Sep. 3, 2025, which claims priority from and the benefit of Korean Patent Application No. 10-2024-0120761, filed on Sep. 5, 2024, Korean Patent Application No. 10-2024-0182522, filed on Dec. 10, 2024, and Korean Patent Application No. 10-2025-0123021, filed on Sep. 1, 2025, each of which is hereby incorporated by reference for all purposes as if fully set forth herein.

### BACKGROUND

### Field

Embodiments of the invention relate generally invention to a system for predicting physical properties and a method therefore, and more particularly, to a system for predicting physical properties, capable of predicting physical properties of a material by separating interatomic interactions constituting the material on a multiscale basis and processing and integrating respective information, and a method therefor.

### Discussion of the Background

Recently, artificial intelligence (AI) technology has been drawing attention throughout the society as it shows advanced development trends. AI refers to computers performing human-specific intellectual capabilities with high-level capability, such as “computer brains that execute works that can be accomplished with human intelligence,” “engineering and science that creates intelligent machines,” and “a series of algorithmic systems designed to think, sense, and act like humans.”

AI is being introduced as providing highly integrated smart spaces when used together with augmented reality, the “Internet of Things”, edge computing, digital twins, and the like, and is being emphasized as a core new technology that will lead the era of the “Fourth Industrial Revolution”. In addition, AI is drawing attention as a next-generation growth engine capable of evolving industrial ecosystems beyond solving standardized problems, and is being actively applied not only to IT, medical care, agriculture, energy, automobiles, and robots, but also to knowledge service industries such as distribution, finance, law, education, real estate, advertisement, and telecommunications. In other words, all existing systems are preparing for a new era by combining with AI, from industries that promote convenience or improvement in real life to all aspects of culture and arts in the society.

As product development methods have diversified in recent years, the development of new materials that can be used in product manufacturing has been actively progressing. Such materials have a significant impact on product characteristics, and sometimes, the physical properties of materials are factors that determine the characteristics of manufactured products. Therefore, in order to more efficiently develop and mass-produce materials used in product manufacturing, it may be essential to predict and analyze the physical properties of such materials.

Traditionally, a method has been used in which multiple materials are developed to confirm the characteristics of manufactured products according to material properties, the properties of each material are confirmed, and then the materials are applied to final products in a pilot scale to confirm their characteristics. However, such conventional methods require significant cost and time for developing materials and confirming their properties, and have the problem that it is difficult to find materials having optimal properties.

The above information disclosed in this Background section is only for understanding of the background of the inventive concepts, and, therefore, it may contain information that does not constitute prior art.

### SUMMARY

Embodiments of the invention provide a system for predicting physical properties that subdivides interatomic interactions into multiple scales of local, semi-global, and global scales and processes information in an optimized manner for each scale to maximize the accuracy of predicting physical properties of a material, and a method therefor.

Additional features of the inventive concepts will be set forth in the description which follows, and in part will be apparent from the description, or may be learned by practice of the inventive concepts.

According to one or more embodiments of the invention, a system for predicting physical properties of a material includes: at least one processor; and at least one memory storing instructions, information, or an AI model executed by the at least one processor, and the instructions, information, or AI model executed by the at least one processor includes: a preprocessing module receiving atomic structure data of the material and generating first input data for a local interaction computation, second input data for a semi-global interaction computation, and third input data for a global interaction computation; a local interaction computation portion computing local interactions between a specific atom and neighboring atoms around the specific atom based on the first input data to extract a local interaction feature; a semi-global interaction computation portion computing interactions between all atoms in a unit cell of a crystal based on the second input data to extract a semi-global interaction feature; and a global interaction computation portion computing long-range interactions in consideration of periodicity between unit cells based on the third input data to extract a global interaction feature.

The local interaction computation portion may include a graph neural network, and the first input data that is input to the graph neural network may have a graph structure in which atoms of the material are nodes and atom pairs located within a specific cut-off radius of the material are edges.

The semi-global interaction computation portion may include a transformer, and the second input data that is input to the transformer may include an input sequence listing feature vectors of atoms in the unit cell in order.

The semi-global interaction computation portion may extract the semi-global interaction feature by using a relative distance matrix between all atom pairs in the unit cell as an attention bias of a self-attention computation.

The global interaction computation portion may include another graph neural network, and the third input data that is input to the other graph neural network may be an extended graph structure connected to neighboring atoms beyond a boundary of the unit cell.

The system may further include a physical property prediction portion, and the physical property prediction portion may generate an integrated feature vector by integrating respective feature vectors of the local interaction feature, the semi-global interaction feature, and the global interaction feature.

The physical property predicting portion may predict a target physical property by inputting the integrated feature vector to a multilayer perceptron.

The preprocessing module may obtain the atomic structure data by receiving a crystallographic information file (CIF).

According to yet another embodiment of the invention, a computerized method for predicting physical properties of a material includes: generating input data including first input data for local interaction computation, second input data for semi-global interaction computation, and third input data for global interaction computation by receiving a crystallographic information file (CIF) as atomic structure data of the material; extracting a local interaction feature by computing local interactions between a specific atom and surrounding neighboring atoms thereof based on the first input data; extracting a semi-global interaction feature by computing, based on the second input data, interactions between all atoms in a unit cell of a crystal; extracting a global interaction feature by computing, based on the third input data, long-range interactions considering periodicity between unit cells; and predicting a target property of the material by integrating the local interaction feature, the semi-global interaction feature, and the global interaction feature.

Generating input data may generate, through a preprocessing module, the first input data having a graph structure in which atoms of the material are nodes and atom pairs located within a specific cut-off radius of the material are edges.

Extracting a local interaction feature may extract the local interaction feature through a message passing computation of a graph neural network on the first input data.

Generating input data may generate the second input data including an input sequence listing feature vectors of atoms in the unit cell in order and a relative distance matrix obtained by calculating distances between all atom pairs.

Extracting a semi-global interaction feature may extract the semi-global interaction feature by applying a self-attention mechanism of a transformer architecture to the second input data and uses the relative distance matrix as an attention bias.

Generating input data may generate the third input data of an extended graph structure connected to neighboring atoms beyond a boundary of the unit cell.

Extracting a global interaction feature may apply a graph neural network to the extended graph structure of the third input data to extract the global interaction feature including interactions between unit cells.

Predicting a target property of the material may predict the target property of the material by combining vectors representing each of the local interaction feature, the semi-global interaction feature, and the global interaction feature to generate an integrated feature vector and passing the integrated feature vector through a fully connected neural network.

According to yet another embodiment of the invention, a service system for predicting physical properties of a material includes: a user computing device transmitting an analysis request for atomic structure data of the material and receiving a predicted physical property value; and a server computing system communicatively connected with the user computing device, the server computing system including: a preprocessing module receiving the atomic structure data as input and generating first input data for local interaction computation, second input data for semi-global interaction computation, and third input data for global interaction computation; a multiscale interaction computation portion extracting a local interaction feature based on the first input data, a semi-global interaction feature based on the second input data, and a global interaction feature based on the third input data; and a physical property prediction portion predicting a target physical property of the material by integrating the extracted respective interaction features.

The multiscale interaction computation portion may include: a local interaction computation portion extracting the local interaction feature by applying message passing computation of a graph neural network to the first input data; a semi-global interaction computation portion extracting the semi-global interaction feature by applying self-attention of a transformer to the second input data; and a global interaction computation portion extracting the global interaction feature by applying message passing computation of a graph neural network to the third input data.

The physical property prediction portion may predict the target physical property by combining respective feature vectors representing the local interaction feature, the semi-global interaction feature, and the global interaction feature to generate an integrated feature vector and inputting the integrated feature vector into a multilayer perceptron.

According to yet another embodiment of the invention, an application-specific integrated circuit for predicting physical properties of a material, configured as a functional block, includes: a memory storing information, instructions, and an AI model; and at least one processor requesting access to the memory, and the memory may store an AI model, instructions, or information receiving a crystallographic information file as atomic structure data of the material and generating first input data including first input data for local interaction computation, second input data for semi-global interaction computation, and third input data for global interaction computation; extracting a local interaction feature by computing local interactions between a specific atom and surrounding neighboring atoms thereof based on the first input data; extracting a semi-global interaction feature by computing interactions between all atoms within a unit cell of a crystal based on the second input data; extracting a global interaction feature by computing long-range interactions considering periodicity between unit cells based on the third input data; and predicting a target physical property of the material by integrating the local interaction feature, the semi-global interaction feature, and the global interaction feature.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the inventive concepts.

FIG. 1 is a schematic diagram illustrating an electronic device according to an embodiment of the invention.

FIG. 2 is a schematic diagram illustrating a physical property prediction model according to an embodiment of the invention.

FIG. 3 is a flow chart illustrating a physical property prediction method according to an embodiment of the invention.

FIG. 4 is a schematic diagram for explaining concepts of local interaction, semi-global interaction, and global interaction as well as a unit cell according an embodiment of the invention.

FIG. 5 is a schematic diagram illustrating a material physical property prediction service system using a physical property prediction model according to an embodiment of the invention.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

When an embodiment may be implemented differently, a specific process order may be performed differently from the described order. For example, two consecutively described processes may be performed substantially at the same time or performed in an order opposite to the described order. Also, like reference numerals denote like elements.

For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

As is customary in the field, some embodiments are described and illustrated in the accompanying drawings in terms of functional blocks, units, and/or modules. Those skilled in the art will appreciate that these blocks, units, and/or modules are physically implemented by electronic (or optical) circuits, such as logic circuits, discrete components, microprocessors, hard-wired circuits, memory elements, wiring connections, and the like, which may be formed using semiconductor-based fabrication techniques or other manufacturing technologies. In the case of the blocks, units, and/or modules being implemented by microprocessors or other similar hardware, they may be programmed and controlled using software (e.g., microcode) to perform various functions discussed herein and may optionally be driven by firmware and/or software. It is also contemplated that each block, unit, and/or module may be implemented by dedicated hardware, or as a combination of dedicated hardware to perform some functions and a processor (e.g., one or more programmed microprocessors and associated circuitry) to perform other functions. Also, each block, unit, and/or module of some embodiments may be physically separated into two or more interacting and discrete blocks, units, and/or modules without departing from the scope of the inventive concepts. Further, the blocks, units, and/or modules of some embodiments may be physically combined into more complex blocks, units, and/or modules without departing from the scope of the inventive concepts.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

The expression “configured to” as used throughout the present disclosure may be used interchangeably with, for example, “suitable for,” “having the capacity to,” “designed to,” “adapted to,” “made to,” or “capable of,” depending on the context. The term “configured to” may not necessarily mean “specifically designed to” only in terms of hardware. Instead, in some contexts, the expression “a system configured to” may mean that the system is “capable of” in conjunction with other devices or components. For example, the phrase “a processor configured to perform A, B, and C” may refer to a dedicated processor (e.g., an embedded processor) for performing the operations in question, or a general-purpose processor (e.g., a CPU or application processor) that is capable of performing the operations in question by executing one or more software programs stored in a memory.

Artificial intelligence (AI) is a field of computer engineering and information technology that studies methods for enabling a computer to perform thinking, learning, self-development, and the like that may be performed by human intelligence, and refers to enabling a computer to mimic intelligent behavior of humans. Furthermore, AI does not exist by itself, but has many direct and indirect relationships with other fields of computer science. In particular, attempts to introduce AI elements into various fields of information technology and utilize them for solving problems in those fields have been very actively made in modern times.

Machine learning is a field of AI and is a research field that gives computers the ability to learn without explicit programming. Specifically, machine learning may be referred to as a technology for studying and building systems that perform learning and prediction based on empirical data and improve their own performance, and algorithms therefor. Machine learning algorithms take an approach of building specific models to derive predictions or decisions based on input data, rather than performing strictly defined static program instructions.

Many machine learning algorithms have been developed according to how to classify data in machine learning. Representative examples include decision tree, Bayesian network, support vector machine (SVM), and artificial neural network (ANN). A decision tree is an analysis method that performs classification and prediction by plotting decision rules in a tree structure. A Bayesian network is a model that expresses probabilistic relationships (conditional independence) between a plurality of variables in a graph structure. Bayesian networks are suitable for data mining through unsupervised learning. An SVM is a model of supervised learning for pattern recognition and data analysis, and it is mainly used for classification and regression analysis. An ANN is an information processing system in which a plurality of neurons, called nodes or processing elements, are connected in the form of a layer structure by modeling the operating principles of biological neurons and the connection relationships between neurons.

An ANN is a model used in machine learning, and it is a statistical learning algorithm inspired by biological neural networks (particularly brains in the central nervous system of animals) in machine learning and cognitive science. Specifically, an ANN may generally refer to models in which artificial neurons (nodes) forming a network by synaptic connections change the connection strength of synapses through learning to have problem-solving capability. The ANN may be used interchangeably with the term neural network.

An ANN may include a plurality of layers, and each of the layers may include a plurality of neurons. An ANN may also include synapses that connect neurons to neurons. An ANN may generally be defined by the following three factors: \textcircled{1} connection patterns between neurons of different layers, \textcircled{2} a learning process that updates connection weights, and \textcircled{3} an activation function that generates an output value from a weighted sum of inputs received from a previous layer.

ANNs may include network models such as deep neural networks (DNN), recurrent neural networks (RNN), bidirectional recurrent deep neural networks (BRDNN), multilayer perceptrons (MLP), and convolutional neural networks (CNN), but are not limited thereto.

ANNs are classified into single-layer neural networks and multilayer neural networks according to the number of layers. A general single-layer neural network includes an input layer and an output layer. A general multilayer neural network includes an input layer, one or more hidden layers, and an output layer.

The input layer is a layer that receives external data, and the number of neurons in the input layer is equal to the number of input variables. The hidden layer is located between the input layer and the output layer, receives signals from the input layer, extracts features, and transmits them to the output layer. The output layer receives signals from the hidden layer and outputs output values based on the received signals. Input signals between neurons are multiplied by respective connection strengths (weights) and then summed, and when this sum is greater than the neuron's threshold, the neuron is activated and outputs an output value obtained through an activation function.

A deep neural network including a plurality of hidden layers between an input layer and an output layer may be a representative ANN implementing deep learning, which is a type of machine learning technology. The term “learning” may be used interchangeably with “training.”

The workflow of machine learning consists of a series of processes for collecting data for training and validation, modeling, and training the model, and may include the processes of training data collection, data inspection and exploration, data preprocessing and cleaning, modeling, and training.

### 1. Training Data Collection

The training data applied to training of the learning model of the inventive concepts may be generated using data collected from multiple samples. In the inventive concepts, at least one or more different types of training data sets may be used to train the learning model, and each training data set may further include one or more experiment-based results used as functional labels. At least a portion of the training data sets may be used to train the learning model, and another portion may be used to validate the trained learning model.

Data used in the graph model of one embodiment of the invention may be represented as an adjacency matrix or an adjacency list so as to be expressed in a graph format represented by nodes and edges. The adjacency matrix and the adjacency list represent connection relationships of the graph as a two-dimensional array and a list, respectively.

Data used for “text embedding” may include textual descriptions of crystal structures. In one embodiment, the textual description data may be obtained through a Robocrystallographer package that generates textual descriptions similar to the manner in which actual crystallographers analyze structures. When generating textual descriptions of crystal structures, the Robocrystallographer package indicates symmetry, local environment, and extended connectivity, and such package may include utilities for identifying molecule names, component orientations, heterostructure information, and the like. Alternatively, data used for text embedding may be derived from CIF files.

### 2. Data Inspection and Exploration

Once training data for learning of the learning model is collected, the collected training data may be subjected to inspection and exploration regarding data structure, noise data, and data cleaning methods for machine learning application.

This data inspection and exploration step is referred to as an “Exploratory Data Analysis” (EDA) step, and EDA may be referred to as a process of observing and understanding collected data from various perspectives. Before learning the data, independent variables, dependent variables, variable types, data types of variables, and the like are inspected through visualization, such as graphs, and statistical tests, and the characteristics of the data and inherent structural relationships may be confirmed in advance. By examining the distribution and values of the data through such EDA, the phenomenon represented by the data can be better understood, and potential problems with the data can be discovered. In addition, through the process of inspecting the data from various perspectives, various patterns that may not have been identified in the problem definition step can be discovered, and based on these, existing hypotheses can be modified or new hypotheses can be established. Exploratory data analysis may broadly include a process of exploring outliers in the data and a process of analyzing relationships between data attributes.

The process of exploring outliers is to check whether outliers are present in the data and may include sampling methods, statistical methods, visualization methods, and the like. The sampling method extracts random samples from the data to confirm overall trends and anomalies in the data values. The statistical method may use summary statistics such as mean, median, and mode for confirming the center of the data, or range and variance for confirming the dispersion of the data. The visualization method may utilize probability density functions, histograms, dot plots, word clouds, time series charts, maps, and the like to determine which statistical indicators are appropriate for individual attributes of the collected data. However, when using statistical indicators, it should be noted that since all data values in the set are reflected in the mean, the mean value is affected when there are outliers, but since one value located in the middle is used as the median, representative results may be obtained even in the presence of outliers.

The process of analyzing relationships between data attributes involves finding combinations of attributes that have meaningful correlations with each other within the data, and the relationship analysis may be performed differently according to attribute combinations between qualitative attributes (categorical variables) that may not be expressed numerically but may be arbitrarily quantified and quantitative attributes (numeric variables) that may be quantified. Qualitative-qualitative relationships (categorical-categorical) may display the number of values corresponding to each pair of attribute values using cross tables and mosaic plots, quantitative-qualitative relationships (numeric-categorical) may be observed through statistical values (mean, median, etc.) for each category or visually represented through box plots. Quantitative-quantitative relationships (numeric-numeric) may analyze the association between two attributes through correlation coefficients. It may be confirmed that a correlation coefficient of −1 indicates a negative correlation where two attributes change in opposite directions, 0 indicates no correlation, and 1 indicates a positive correlation where two attributes always change in the same direction. The relationship between two attributes having a correlation coefficient may also exhibit various patterns, which may be visually represented using scatter plots.

### 3. Data Preprocessing and Cleaning

Data that has completed inspection and exploration undergoes data preprocessing to be processed into a form suitable for models for machine learning. Data preprocessing involves cleaning data and transforming it into a form that may be understood by the model, and data preprocessing may generally include handling missing data, outlier removal, data scaling, categorical data encoding, feature selection and extraction, and data transformation. All or part of the detailed processes of data preprocessing may be selectively performed, and a separate machine learning model may be used for data preprocessing.

Handling missing data is processing missing values when they are present in the data, wherein the missing values may be represented as NaN (Not a Number) or null values or may be deleted. As the missing values are filled or deleted within the data, the completeness of the data is improved, and when filling the missing values, an average value, a median value, a mode value, or the like may be used.

Outlier removal is removing outliers from data, which are values that deviate from typical data patterns. Since outliers may degrade model performance, they must be removed or replaced, and outliers may be identified and corresponding rows or columns may be deleted or replaced with other values.

Data scaling is a process of adjusting the size of data, and the range of data may be adjusted through data scaling so that the performance of the model may be improved or the convergence speed may be enhanced. Through data scaling, the characteristics of the data may be adjusted to a similar range, and generally, standardization and normalization may be applied to data scaling. Standardization is a method of converting data into a distribution with a mean of 0 and a standard deviation of 1, mainly using the mean and the standard deviation for conversion, and the standardized value z may be denoted as z=(x−μ)/c (where x is the original value, μ is the mean, and σ is the standard deviation). Normalization is a method of converting the range of data to [0,1] or [−1,1], mainly using the minimum value and the maximum value to transform the data, and the normalized value xₙₒᵣₘ may be denoted as xₙₒᵣₘ=(x−xₘᵢₙ)/(xₘₐₓ−xₘᵢₙ) (where x is the original value, xₘᵢₙ is the minimum value, and xₘₐₓ is the maximum value).

Categorical data encoding is converting categorical variables represented by character strings or integer values that may not be directly input to a model into numerical form that may be input to the model. Generally, one-hot encoding or label encoding may be used to convert categorical variables into numerical form.

Feature selection and extraction is for selecting the most useful features for model learning or extracting new features to improve the performance of the model, and through this process, the complexity of the model can be reduced and overfitting can be prevented.

Data transformation is for transforming data to extract new information or enable the model to better understand the data, and it may include tokenization of text data or preprocessing of image data. Through data transformation, useful features may be extracted from original data, or data may be transformed into an appropriate form to improve the performance of the model.

Through the above-described data preprocessing, the effects of improving performance and ensuring stability of the machine learning model can be achieved.

When training the learning model according to an embodiment of the invention, a process of preprocessing information expressed in natural language and a process of training a language model based on the preprocessed data may be performed.

### 3-1. Text Preprocessing for Large Language Models

When the collected data is not preprocessed as needed, tokenization, cleaning, and normalization may be performed according to the intended use of the data.

Tokenization refers to the operation of dividing given data into units called tokens, and the units of tokens may generally be defined as meaningful units. Tokenization may broadly include “word tokenization” and “sentence tokenization”.

“Word tokenization” refers to a case where tokens are based on words, and here the word may include word phrases and character strings having meaning in addition to word units. “Word tokenization” refers to separating words based on symbols such as spaces or punctuation marks, for example, periods, commas, question marks, semicolons, exclamation marks, and the like. However, when all punctuation marks and special characters are removed during the tokenization operation, tokens may lose their meaning, and thus, a precise algorithm for tokenization may be required. For example, when a word itself includes punctuation marks or special characters having meaning are used, the problem may not be solved simply by removing such punctuation marks or special characters. Therefore, tokenization rules such as “Penn Treebank Tokenization” rules may be applied during tokenization.

“Sentence tokenization” refers to dividing text into sentence units. Typically, when data has not been cleaned, a corpus is not divided into sentence units, so sentence tokenization may be required to suit the intended use of the corpus. For such sentence tokenization, various rules may be defined depending on the language used and how special characters are used within the corpus.

The operation of classifying tokens according to their intended use is called “tokenization”, and before and after the tokenization operation, cleaning and normalization are performed on the text data according to the intended use. Cleaning involves removing noise data, and normalization involves integrating words with different representation methods to convert them into the same word.

The cleaning operation may be performed before the tokenization operation to exclude parts that interfere with the tokenization operation, but it may also be continuously and repeatedly performed to remove noise that still remains after the tokenization operation. The noise data removed in the cleaning operation consists of characters that have no meaning, and methods for removing unnecessary words include stop word removal and methods for removing words with low occurrence frequency and short-length words.

The normalization operation includes integration of words with different notations based on rules, uppercase/lowercase integration, and the like. Uppercase/lowercase integration is a normalization method that may reduce the number of words in English-language texts. In English, uppercase letters are used only in specific situations such as at the beginning of sentences, and most text is written in lowercase letters. Therefore, the uppercase/lowercase integration operation may mainly consist of a lowercase conversion operation that converts uppercase letters to lowercase letters.

In order to process natural language in a computing system, a preprocessing operation of converting text into numerical values is required, and to this end, an operation of mapping each word of the text to a unique integer is performed. Such a mapping process may utilize techniques such as “integer encoding”, “padding”, and “one-hot encoding”.

“Integer encoding” is one method of assigning integers to words, and in this method, a vocabulary in which words are arranged in order of frequency is created, and integers are sequentially assigned from lower numbers in order of highest frequency. Integer encoding performs sentence tokenization from text data including multiple sentences as well as word tokenization in parallel with cleaning and normalization operations. At this time, words are converted to lowercase to standardize the number of words, and words may be deleted based on stop words and word length criteria. Through this process, words may be recorded as keys and the frequency of each word may be recorded as values. After arranging words in the text in order of frequency, integer encoding may be performed by assigning integers to words having high frequency.

“Padding” is an operation for arbitrarily equalizing the lengths of sentences having different lengths within text. A computing system may bundle sentences having the same length as a single matrix to enable parallel operations. In other words, to perform parallel operations of the computing system, the integer encoding results of sentences having different lengths within the text may be arbitrarily filled with ‘0’ to equalize the lengths of the sentences. In other words, the longest sentence is identified in the word set for which integer encoding has been completed, and “0” may be added to the integer matrix to correspond to the length of the longest sentence. The computing system may recognize sentences having the same length as one matrix to perform parallel processing, wherein the computing system may ignore the “0” word which is recognized as meaningless. Filling data with a specific value to adjust the shape of the data in this manner is called padding, and when the number “0” is used for length adjustment, it is referred to as zero padding.

“One-hot encoding” is a vector representation method for words in which the size of the word set serves as the dimension of the vector, a value of 1 is assigned to the index of the word to be represented, and 0 is assigned to other indices. The vector represented in this manner is called a “one-hot vector”. “One-hot encoding” includes integer encoding and an index assignment process. After integer encoding is performed to assign a unique integer to each word, the unique integer of the word to be represented is regarded as an index, and “1” is assigned to the corresponding position, while “0” is assigned to the positions of indices of other words. However, one-hot encoding has disadvantages in that the space required to store vectors increases as the number of words increases (increase in vector dimension) and that the similarity between words may not be determined. To overcome these disadvantages, techniques for vectorizing words into a multidimensional space by reflecting the latent meaning of words include count-based vectorization methods such as latent semantic analysis (LSA), prediction-based vectorization methods such as NNLM, RNNLM, Word2Vec and FastText, and the GloVe method which uses both count-based and prediction-based approaches.

In order for computers to understand and process text, the text must be appropriately converted into numbers. Since the performance of natural language processing significantly varies depending on the method of representing words, many technologies for numericalizing words have been proposed. Currently, word embedding methods that vectorize each word through ANN learning are most widely used.

“Word embedding” is a method of representing words as vectors, converting words into dense representations. The result derived through the word embedding process is called a “dense vector” or an “embedding vector”. Word embedding methodologies include LSA, Word2Vec, FastText, GloVe, and the like.

### 4. Modeling and Training

An ANN may be trained using training data. Here, “training” may refer to a process of determining parameters of the ANN using training data to achieve objectives such as classification, regression analysis, or clustering of input data. Representative examples of parameters of the ANN include weights assigned to synapses and biases applied to neurons.

The ANN trained by training data may classify or cluster input data according to patterns possessed by the input data. An ANN trained using training data may be referred to as a trained model herein.

The following describes methods of training ANNs. Methods of training ANNs may be broadly classified into supervised learning, unsupervised learning, semi-supervised learning, and reinforcement learning.

Supervised learning is a method of machine learning for inferring a function from training data. Among such inferred functions, those that output continuous values are called regression analysis, and those that predict and output the class of input vectors are called classification.

In supervised learning, an ANN is trained with training data for which labels are provided. Here, a label refers to the correct answer (or result value) that the ANN should infer when training data is input to the ANN Herein, the correct answer (or result value) that the ANN should derive when training data is input is referred to as a label or labeling data. Additionally, herein, setting a label on training data for training of the ANN is referred to as “labeling the training data with labeling data”. In this case, the training data and the label corresponding to the training data constitute one training set, and they may be input to the ANN in the form of a training set.

The training data represents a plurality of features, and that the training data is labeled with a label may mean that the features represented by the training data are labeled. In this case, the training data may represent the features of the input object in a vector form. The ANN may infer a function for the association relationship between the training data and the labeling data by using the training data and the labeling data. In addition, parameters of the ANN may be determined (optimized) through evaluation of the function inferred by the ANN.

Unsupervised learning is a type of machine learning in which no label is given to the training data. Specifically, unsupervised learning may be a learning method in which an ANN is trained to find and classify patterns in the training data itself, rather than an association relationship between the training data and a label corresponding to the training data. Examples of unsupervised learning include clustering or independent component analysis.

Examples of ANNs using unsupervised learning include a generative adversarial network (GAN) and an autoencoder (AE).

A GAN is a machine learning method in which two different AIs, a generator and a discriminator, compete to improve performance. In this case, the generator is a model that creates new data, and it may generate new data based on original data. In addition, the discriminator is a model that recognizes patterns of data, and it may serve to discriminate whether input data is original data or new data generated by the generator. In addition, the generator may receive and learn from data that failed to fool the discriminator, and the discriminator may receive and learn from the data from the generator that fooled the discriminator. Accordingly, the generator may evolve to fool the discriminator as well as possible, and the discriminator may evolve to well distinguish between the original data and the data generated by the generator.

An autoencoder is a neural network that aims to reproduce an input itself as an output. The autoencoder includes an input layer, at least one hidden layer, and an output layer. In this case, since the number of nodes of the hidden layer is less than the number of nodes of the input layer, the dimensions of the data are reduced, and thus compression or encoding is performed. In addition, the data output from the hidden layer enters the output layer. In this case, since the number of nodes of the output layer is greater than the number of nodes of the hidden layer, the dimensions of the data are increased, and thus, decompression or decoding is performed.

The autoencoder adjusts the connection strength of neurons through learning, so that the input data is represented as hidden layer data. In the hidden layer, information is represented by a smaller number of neurons than in the input layer, but that the input data may be reproduced as the output may mean that the hidden layer discovers and represents hidden patterns from the input data.

Semi-supervised learning is a type of machine learning, and it may refer to a learning method that uses both training data with a given label and training data without a given label. One technique of semi-supervised learning is inferring a label of training data without a given label and then performing learning using the inferred label, and such a technique may be effectively used when the cost involved in labeling is high.

Reinforcement learning is based on a theory that an agent may find the best way through experience without data, given an environment in which the agent may determine what action to take at every moment. Reinforcement learning may be performed mainly by a Markov Decision Process (MDP). To describe the Markov decision process, first, an environment in which information necessary for the agent to take a next action is configured is given, second, how the agent should act in the environment is defined, third, what the agent does well is rewarded and what it does poorly is penalized, and fourth, an optimal policy is derived through iterative experience until a future reward reaches a maximum point.

The structure of an ANN is specified by the model configuration, activation function, loss function or cost function, learning algorithm, optimization algorithm, and the like, and hyperparameters may be preset before learning, and thereafter model parameters may be set through learning to specify the content.

For example, elements that determine the structure of an ANN may include the number of hidden layers, the number of hidden nodes included in each hidden layer, input feature vectors, target feature vectors, and the like.

The hyperparameter includes various parameters that need to be initially set for learning, such as an initial value of a model parameter. In addition, the model parameter includes various parameters to be determined through learning. For example, the hyperparameters may include an initial value of the weight of the nodes, an initial value of the bias of the nodes, a mini-batch size, a number of learning iterations, a learning rate, and the like. In addition, the model parameters may include a weight of the nodes, a bias of the nodes, and the like.

The loss function may be used as an indicator (criterion) for determining optimal model parameters in the learning process of an ANN. In ANNs, learning refers to a process of manipulating model parameters to reduce the loss function, and the purpose of learning may be viewed as determining model parameters that minimize the loss function. The loss function may mainly use mean squared error (MSE) or cross entropy error (CEE), and the invention is not limited thereto. The CEE may be used when a correct answer label is one-hot encoded. One-hot encoding is an encoding method in which a correct answer label value is set to 1 only for a neuron corresponding to a correct answer, and a correct answer label value is set to 0 for neurons that are not correct answers.

In machine learning or deep learning, a learning optimization algorithm may be used to minimize the loss function. Examples of the learning optimization algorithm include gradient descent (GD), stochastic gradient descent (SGD), Momentum, Nesterov Accelerated Gradient (NAG), Adagrad, AdaDelta, RMSProp, Adam, Nadam, and the like.

GD is a technique of adjusting model parameters in a direction that reduces the loss function value by considering the gradient of the loss function in the current state. The direction in which the model parameters are adjusted is referred to as a “step direction”, and the magnitude of the adjustment is referred to as a “step size”. In this case, the step size may refer to a “learning rate”. In GD, the gradient may be obtained by partially differentiating the loss function with respect to each model parameter, and the model parameters may be updated by changing them by the learning rate in the obtained gradient direction.

SGD is a technique in which the training data is divided into mini-batches, and GD is performed for each mini-batch to increase the frequency of gradient descent.

“Adagrad”, “AdaDelta”, and “RMSProp” are techniques for adjusting the step size in SGD to increase optimization accuracy. “Momentum” and “NAG” in SGD are techniques for adjusting the step direction to increase optimization accuracy. “Adam” is a technique for adjusting both the step size and the step direction by combining Momentum and RMSProp to increase optimization accuracy. “Nadam” is a technique for adjusting both the step size and the step direction by combining NAG and RMSProp to increase optimization accuracy.

The learning speed and accuracy of an ANN are greatly dependent on the structure of the ANN and the type of learning optimization algorithm as well as hyperparameters. Therefore, in order to obtain a good learning model, it is important not only to determine an appropriate ANN structure and learning algorithm, but also to set appropriate hyperparameters.

Typically, hyperparameters are experimentally set to various values while training the ANN, and as a result of the training, they are set to optimal values that provide stable learning speed and accuracy.

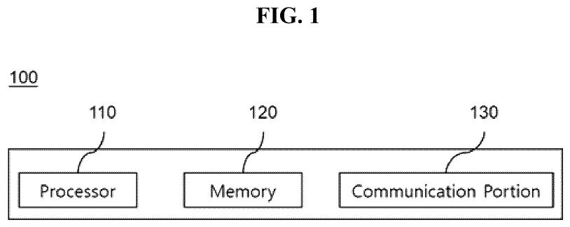

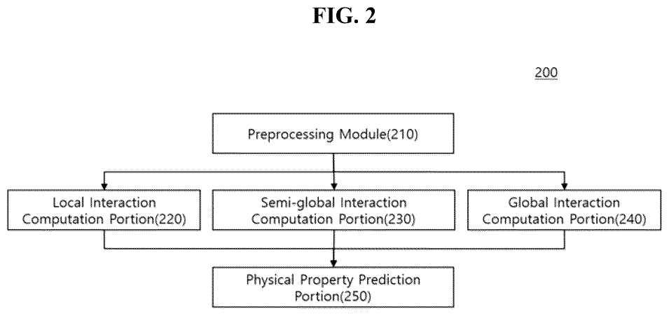

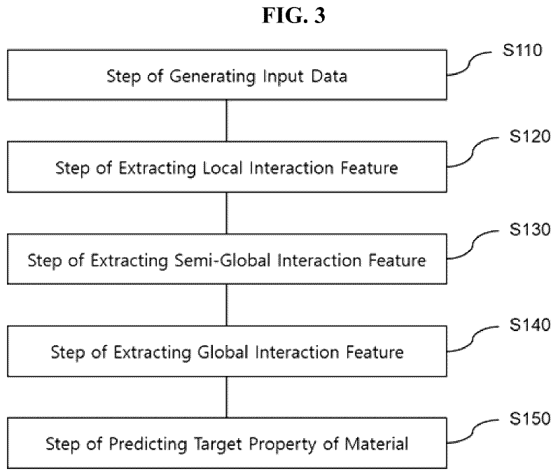

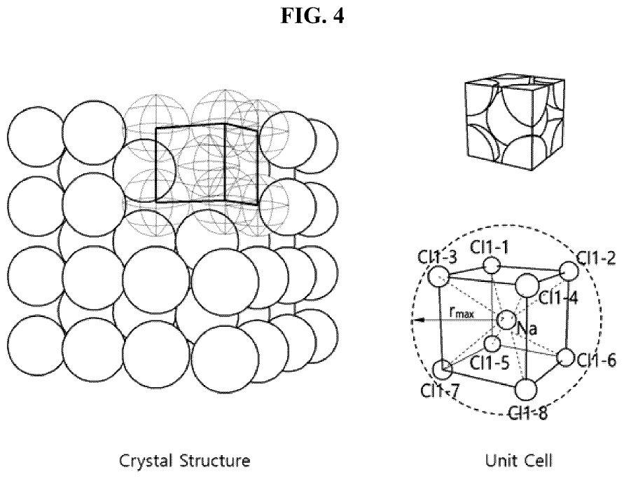

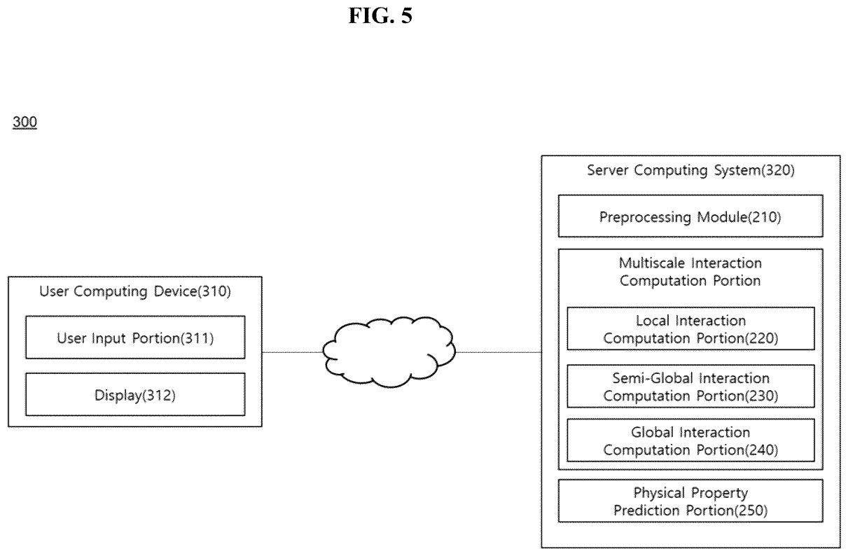

FIG. 1 is a schematic diagram illustrating an electronic device according to one embodiment of the invention, FIG. 2 is a schematic diagram illustrating a physical property prediction model according to an embodiment of the invention, FIG. 3 is a flow chart illustrating a physical property prediction method according to one embodiment of the invention, FIG. 4 is a schematic diagram for explaining concepts of local, semi-global, and global interaction as well as a unit cell according to one embodiment of the invention, and FIG. 5 is a schematic diagram illustrating a material physical property prediction service system using the physical property prediction model according to one embodiment of the invention.

As shown in FIG. 1, the electronic device 100 according to embodiments of the invention may include a processor 110, a memory 120, and a communication portion 130. The electronic device 100 is a basic component for performing a computing environment, and in other embodiments, the electronic device 100 may be implemented by additionally or alternatively including some other components, may be implemented as a single or plurality of entities, or may be implemented with only some of the disclosed components. At least some of the components inside or outside the electronic device 100 may be connected to each other through a BUS, General Purpose Input/Output (GPIO), Serial Peripheral Interface (SPI), Mobile Industry Processor Interface (MIPI), or the like to transmit and receive data or signals.

The processor 110 may refer to a set of one or more processors unless the context clearly indicates otherwise, and it may control the components of the processor 110 and the electronic device 100 by driving at least software (e.g., instructions, programs, etc.) stored in the memory 120. In addition, the processor 110 may perform various operations, such as computations, processing, data generation, or processing, and it may read data from the memory 120 or store data in the memory 120. The processor 110 may be configured with at least one core and may include processors for data analysis, machine learning (ML), or deep learning (DL), such as a central processing unit (CPU), a general purpose graphics processing unit (GPGPU), or a tensor processing unit (TPU). The processor 110 may read software stored in the memory 120 to perform data processing for machine learning (or deep learning) of the invention. According to an embodiment of the invention, the processor 110 may perform computations for training a neural network. The processor 110 may perform calculations for training a neural network, such as processing input data for training in deep learning, feature extraction from the input data, error calculation, and updating weights of the neural network using backpropagation. At least one of the CPU, the GPGPU, and the TPU of the processor 110 may process training of the neural network model. For example, the CPU and the GPGPU may together process training of a neural network model and data classification using the neural network model. In addition, in one embodiment of the invention, at least one processor 110 of the electronic device 100 may be used together to process training of a neural network model and data classification using the neural network model.

The memory 120 is for storing various data, and the data may include software (e.g., instructions, programs, etc.) as data obtained, processed, or used by at least one component of the electronic device 100. The memory 120 may refer to a set of one or more memories unless the context clearly indicates otherwise, and it may include at least one type of storage medium among flash memory type, hard disk type, multimedia card micro type, card type memory (e.g., SD or XD memory, etc.), RAM, SRAM (Static Random Access Memory), ROM, EEPROM (Electrically Erasable Programmable Read-Only Memory), PROM (Programmable Read-Only Memory), magnetic memory, magnetic disk, optical disk, and web storage performing a storage function on the Internet. Instructions, programs, or software stored in the memory 120 may be used to refer to an operating system for controlling components of the electronic device 100, applications, or middleware that provides various functions to applications so that the applications may utilize the components of the electronic device 100. In one embodiment, when the processor 110 performs a specific computation, the memory 120 may store instructions performed by the processor 110 and corresponding to the specific computation.

The communication portion 130 performs wireless or wired communication between the electronic device 100 and another device (e.g., a user terminal or another server). The communication portion 130 may use wireless communication systems according to schemes such as eMBB, URLLC, MMTC, LTE, LTE-A, NR, UMTS, GSM, CDMA, WCDMA, TDMA, FDMA, OFDMA, SCFDMA, WiBro, WiFi, Bluetooth, NFC, GPS, or GNSS. In addition, the communication portion 130 may use various wired communication systems such as USB, HDMI, Recommended Standard-232 (RS-232), Plain Old Telephone Service (POTS), Public Switched Telephone Network (PSTN), x Digital Subscriber Line (xDSL), Rate Adaptive DSL (RADSL), Multi Rate DSL (MDSL), Very High Speed DSL (VDSL), Universal Asymmetric DSL (UADSL), High Bit Rate DSL (HDSL), and Local Area Network (LAN). In one embodiment of the invention, the communication portion 130 may be configured regardless of communication modes such as wired communication and wireless communication, and it may be configured as various communication networks such as a Personal Area Network (PAN), Wide Area Network (WAN), or the like. In addition, the communication network may be a known World Wide Web (WWW), or it may use wireless transmission technology used for short-range communication such as Infrared Data Association (IrDA) or Bluetooth.

The electronic device 100 according to one embodiment of the invention may further include an input/output portion (not shown). The input/output portion may be configured to be separated into an input portion and an output portion, but alternatively, the input/output portion may have an integrated configuration. The input portion serves as a means for data input and may be configured in various types. For example, the input portion may be configured to receive user input. The input portion may be configured to receive user input from a user terminal. Here, “receiving an input” may mean receiving an input signal (or selection signal) corresponding to the user's input based on the input made by the user through the input portion configuration provided in the user terminal. The input portion may also be referred to as a user interface module. The input portion may include a touch screen, a computer mouse, a keyboard, a keypad, a touch pad, a trackball, a joystick, a voice recognition module, or other similar devices. However, the invention does not limit the type of input portion. In addition, in the invention, the input portion does not necessarily mean a hardware means, but it may be understood as a pathway for receiving input from a user. Here, the user input may include documents, text, images (or videos), or the like. Next, the output portion may output information through an output portion configuration (for example, a display portion, touch screen, or the like) provided in the user terminal or computing device. The output portion does not necessarily mean a hardware means, but it may be understood as a pathway for outputting results to the user.

The electronic device 100 according to an embodiment of the invention may execute software that configures a physical property prediction system or configures a physical property prediction method.

One embodiment of the invention may utilize both the local interaction representation capability, which is an advantage of GNN, and the long-range interaction representation capability, which is an advantage of the transformer, while subdividing them into physically meaningful multiple scales to maximize modeling accuracy. Specifically, the physical property prediction system according to one embodiment of the invention may divide interatomic interactions in a material into three scales, extract features by applying an optimized architecture to each scale, and then integrate them to predict physical properties. Here, the three types of interactions between atoms in a material may include local interaction, semi-global interaction, and global interaction. Referring to FIG. 4, local interaction may refer to strong chemical bonds and interactions between a specific atom and neighboring atoms around it, semi-global interaction may refer to interactions between all atoms within a unit cell, which is the basic unit of a crystal structure, and global interaction may refer to long-range interactions between unit cells or between atoms in an extended lattice considering the characteristics of the crystal structure in which unit cells are periodically repeated. Here, a unit cell is the smallest repeating unit that forms a crystal structure, and when such unit cells are stacked in a three-dimensional space, the entire crystal structure may be completed. In other words, the unit cell serves as a core blueprint containing all information of the entire crystal.

As shown in FIG. 2, the physical property prediction model 200 of the physical property prediction system according to one embodiment of the invention may include a preprocessing module 210, a local interaction computation portion 220, a semi-global interaction computation portion 230, a global interaction computation portion 240, and a physical property prediction portion 250.

The preprocessing module 210 is a processing module that receives atomic structure data of a material to learn or a material to be predicted and converts it into a form that may be processed by the system or learning model. The physical property prediction model 200 according to one embodiment of the invention may receive input of data in a format describing the atomic structure of a material with three-dimensional coordinates, for example, Crystallographic Information File (CIF) data.

CIF is a standard text file format for exchanging and storing crystallographic data, and it is designed to enable standardization of data in the field of crystallography so that computing devices may easily interpret the atomic structure of materials. A CIF file is structured in a simple text-based format according to specific rules, and the main components may include data blocks, data names, data values, and loop structures. The data block is for specifying a plurality of crystal structures included within a single CIF file. One CIF file may include one or more data blocks, and each block may use the name of a specific compound. In other words, multiple crystal structure information may be included in one file. The data name (or tag) is a standardized name for identifying each data item; for example, the data name indicating the a-axis length of a unit cell may be described as _cell_length_a. The data value is an actual value corresponding to the data name, and the value may be separated from the data name by a space. The loop structure is for efficiently representing repetitive data, such as multiple atomic coordinates. The loop structure uses, for example, a loop_ directive, and from the line following loop_, multiple data names may be listed, and below that, values corresponding to each name may be listed in order.

The CIF input data in this format may include the type of each atom, three-dimensional information, and information of a unit cell containing them, and the preprocessing module 210 may extract such information from such input data.

Alternatively, in another embodiment of the invention, the input data for the crystal structure may be obtained through a Robocrystallographer package that generates text descriptions similar to how actual crystallographers analyze structures. The Robocrystallographer package displays symmetry, local environment, and extended connectivity when generating text descriptions for crystal structures, and such package may include utilities for identifying molecule names, component orientations, heterostructure information, and the like. For example, when SnO₂ is given as input, the Robocrystallographer used in one embodiment may output text such as “SnO₂ is Rutile structured and crystallizes in the tetragonal P4_2/mnm space group. The structure is three-dimensional. Sn(1) is bonded to six equivalent O(1) atoms to form a mixture of edge and corner-sharing SnO6 octahedra. The corner-sharing octahedral tilt angles are 51°. All Sn(1)-O(1) bond lengths are 2.09 Å. O(1) is bonded in a trigonal planar geometry to three equivalent Sn(1) atoms.” Such text descriptions may include a wide range of information including global properties (e.g., space group and crystal type), local detailed information (e.g., bond lengths and coordination environments), and semi-global properties (e.g., connectivity and structural arrangements).

In one embodiment of the invention, the preprocessing module 210 may pre-process the CIF input data so as to be processable by respective computation portions. In other words, the preprocessing module 210 may generate input data in different forms that may respectively be processed by the local interaction computation portion 220, the semi-global interaction computation portion 230, and the global interaction computation portion 240.

First, the preprocessing module 210 may convert (generate) input data of a graph data structure for GNN. In one embodiment of the invention, the local interaction computation portion 220 and the global interaction computation portion 240 may be configured as a GNN, but unit cell information may be further added to the data input to the global interaction computation portion 240 so that an overall graph including the periodicity of the crystal structure is generated.

The information extracted by the preprocessing module 210 may be converted into a graph data structure having atoms as nodes and interatomic distances or interatomic bonds as edges. The graph data converted by the preprocessing module 210 may then be passed to subsequent computation portions. Here, each node may have an initial feature vector representing element-specific features such as atomic number, atomic weight, electronegativity, and the like. This initial feature vector may be converted into a high-dimensional vector through an atom embedding layer. Additionally, edges, which are interatomic relationships, may be generated for atom pairs within a specific cut-off radius. In other words, edges may have geometric information, such as distance between two atoms, direction, and the like, as features.

Unit cell information may be further added to the data input to the global interaction computation portion 240 configured as a GNN so that an overall graph including the periodicity of the crystal structure is generated.

To this end, the preprocessing module 210 may generate a graph representing the overall structure including the periodicity of the crystal when converting input data for the GNN of the global interaction computation portion 240. The preprocessing module 210 may connect neighboring atoms based on long distances, for example, with a large cut-off of 8 to 10 Å. At this time, periodic boundary conditions (PBC) may be applied to create edge indices including all neighbors beyond the unit cell boundary. In addition, the preprocessing module 210 may include information indicating a lattice in which the neighbors are located, along with the interatomic distances. Here, the PBC is a method of representing the entire crystal structure that is infinitely repeated with only one unit cell, which is the smallest repeating unit.

Furthermore, the preprocessing module 210 may convert input data for a transformer of the semi-global interaction computation portion 230. The preprocessing module 210 may generate a sequence of atoms and all inter-atomic distance relationship information from the CIF file. The input sequence is a tensor that lists initial atomic feature vectors in order, and the distance matrix between all atom pairs within the unit cell may serve as attention bias. In other words, the preprocessing module may perform steps of parsing, characterization, and sequence generation to convert input data for the transformer. Parsing is a process of extracting crystal structure information from a CIF file by reading the CIF file to extract atomic types (element symbols), atomic coordinates (three-dimensional coordinates), and lattice parameters (unit cell size and angles). Characterization is a process of converting the extracted information into numbers that the transformer may understand, and in this process, each atom is treated as a token and a vector representing this token is generated. The preprocessing module may generate vectors for key atomic features such as atomic number, periodic table, electronegativity, ionization energy, atomic radius, covalent bond radius, and the like and calculate Euclidean distances between all atom pairs to produce a relative distance matrix. Through this, three-dimensional spatial information of the atoms may be generated. The preprocessing module may combine the characterized information to convert the transformer input data. The preprocessing module may list atomic feature vectors of each atom in the crystal structure in order so as to form an input sequence, and it may add the relative distance matrix, which is three-dimensional position information of the atoms, as bias to the attention matrix.

The preprocessing module 210 may convert and generate input data of the transformer including the atomic feature vector tensor as main input and the relative distance matrix as attention bias through the above-described process.

The local interaction computation portion 220 is for computing local interaction information at the atomic level, specifically for computing direct interactions between a specific atom within a material and its neighboring atoms, such as interatomic chemical bonds and van der Waals forces, which are direct interactions between adjacent atoms. In other words, the local interaction computation portion 220 may compute interaction information between an atom and neighboring atoms within a specific cut-off radius, and in an embodiment of the invention, a GNN may be applied to the local interaction computation portion 220.

GNN models, particularly those of the Message Passing Neural Network (MPNN) family, repeat a process in which each node (atom) propagates its feature vector in the form of a ‘message’ to neighboring nodes while simultaneously receiving messages from neighbors to update its state. Through this process, chemical and structural environment information of surrounding atoms may be progressively encoded in the feature vector of each atom. In the GNN according to one embodiment of the invention, feature vectors may be updated by repeatedly performing message passing computations on graph data in which atoms transferred from the preprocessing module 210 serve as nodes and atom pairs within a certain distance (cut-off radius) serve as edges. In other words, in the GNN, at each computation step, an atom (node) collects information from neighboring atoms (neighboring nodes) and bonds (edges) to update its feature vector. Through this process, local interaction features may be extracted that intricately embed the local chemical environment at the atomic level and bonding features. The GNN may enable atoms to progressively integrate information from a wider range of neighboring atoms by repeating message passing computations across multiple layers. In other words, the GNN may represent local chemical environments based on geometric relationships such as interatomic distances and angles as embedding vectors by aggregating the feature vector of an atom with feature vectors of neighboring atoms and repeating this process across multilayer layers. The features derived through the local interaction computation portion 220 may include the most fundamental information that determines material properties.

Referring to FIG. 4, the interatomic local interaction computed by the local interaction computation portion 220 according to one embodiment of the invention may refer to interactions within the local atomic environment of a unit cell centered on a specific atom, such as interactions between a Na atom and Cl atoms (Cl1-1, Cl1-2, etc.) within a cut-off radius (rₘₐₓ). In other words, such interatomic local interaction computations in the local atomic environment may derive properties directly related to the formation of interatomic chemical bonds.

The semi-global interaction computation portion 230 is for computing structural feature information at the unit cell level by comprehensively considering interactions between all atom pairs present within a unit cell. In other words, the semi-global interaction computation portion 230 may calculate structural feature information of the entire unit cell beyond the local environment of individual atoms in the material by modeling atomic arrangements and interactions throughout the entire unit cell, which is the basic structural unit of the material. Through this, the semi-global interaction computation portion 230 may compute properties such as symmetry and density of the entire unit cell, including interactions between relatively distant atoms without direct bonding, that is, interactions between distant atoms beyond the cut-off radius (GNN cut-off radius) shown in FIG. 4. In one embodiment of the invention, a transformer architecture may be applied to the semi-global interaction computation portion 230 to comprehensively consider interactions between all atoms within the unit cell.

As the semi-global interaction computation portion 230 according to one embodiment of the invention, the transformer may set all atoms within a unit cell as a sequence and apply a self-attention mechanism to this atomic sequence. The semi-global interaction computation portion 230 may calculate attention weights (attention scores) through the transformer's self-attention mechanism to determine how much one atom in the sequence, that is, one atom in the unit cell, is related to all other atoms. As these relationship weights are calculated, interaction relationships between all atoms in the unit cell may be captured. In other words, the self-attention mechanism, which is the core of the transformer, may generate “Query,” “Key,” and “Value” vectors for all input atoms (tokens) and calculate attention weights by computing similarities of all query-key pairs. These attention weights indicate how much attention each atom should pay to all other atoms in the unit cell, and by applying these weights to value vectors to calculate weighted sums, the feature vector of each atom may be updated.

In this manner, the semi-global interaction computation portion 230 of one embodiment of the invention may learn features of the unit cell that are difficult for GNN to capture, such as symmetry, orientation (coordination environment), and electrical interactions of the unit cell. In other words, unlike GNN, the semi-global interaction computation portion 230 of one embodiment directly models relationships between all atoms without distance constraints, thereby effectively learning interactions across the entire unit cell.

The global interaction computation portion 240 is for computing long-range interactions between unit cells that are periodically repeated within a material crystal structure. In other words, the global interaction computation portion 240 of one embodiment of the invention may calculate long-range interaction information between a reference unit cell and neighboring unit cells or within an extended supercell. The global interaction computation portion 240 of one embodiment of the invention may capture interactions that occur when unit cells are repeated in a three-dimensional space, by modeling long-range interactions that occur due to the periodicity of the crystal structure beyond the unit cell.

Referring to FIG. 4, the global interaction captured through the global interaction computation portion 240 according to one embodiment refers to long-range interactions occurring between a reference unit cell and neighboring unit cells that are repeatedly arranged by PBCs, and the interactions may play a very important role in determining the bulk properties of the material.

In one embodiment of the invention, a GNN may be utilized as the global interaction processor 240, but in another embodiment, a transformer may be applied as the global interaction processor 240.

In one embodiment, when a GNN is used as the global interaction processor 240, the GNN may regard a “supercell” constructed by replicating multiple unit cells as one large graph and apply the GNN thereto to model long-range interactions. In other words, the GNN-based global interaction processor 240 may construct a larger-scale graph that regards the unit cell itself as one node of the graph or may apply PBCs to extend the graph such that atoms in the original unit cell interact with atoms of other adjacent unit cells to perform computations. When the GNN is applied to this extended graph, message passing propagates beyond the boundaries of the unit cell, thereby naturally learning inter-lattice interactions.

In another embodiment of the invention, when a transformer is used as the global interaction computation portion 240, a special token such as a “class token” may be introduced to intensively learn global features of the entire crystal structure. In other words, in another embodiment, a “class token” is added to a set of atoms in a unit cell input to the transformer, and this class token may be trained to aggregate global information of the entire lattice while passing through transformer layers. After such a class token is introduced, the global interaction computation portion 240 may optionally reflect periodicity of the unit cells in positional encoding. The global interaction computation portion 240 may apply self-attention to all sets of atoms in an extended supercell to calculate the most comprehensive range of interactions. Through such a process, the global interaction computation portion 240 may extract global interaction features related to macroscopic-scale physical phenomena such as lattice strain and phonon vibration of the entire crystal.

As shown in FIG. 2, the physical property prediction model 200 according to one embodiment of the invention may further include a physical property prediction portion 250 for combining feature vectors (embeddings) output from respective computation portions into one vector and predicting a target physical property. In one embodiment of the invention, the physical property prediction portion 250 may combine the local/semi-global/global interaction feature vectors respectively output from the local interaction computation portion 220, the semi-global interaction computation portion 230, and the global interaction computation portion 240 into one integrated feature vector through concatenation or element-wise summation computations. Such an integrated feature vector includes all microscopic/macroscopic/multilayer interaction information of the material. The physical property prediction model 200 may transfer the integrated feature vector to a neural network such as an MLP including at least one fully-connected layer and map the integrated feature vector to final physical property values such as formation energy, band gap, elastic modulus, and the like. Through this process, the physical property prediction model 200 according to one embodiment of the invention may perform a task of predicting a target physical property of the material using a two-layer MLP.

As shown in FIG. 3, a physical property prediction method according to an embodiment of the invention is executed by a computer, and may include an input data generation step S110 of receiving atomic structure data of a material as input and generating input data for each of a local interaction computation, a semi-global interaction computation, and a global interaction computation; a local interaction feature extraction step S120 of extracting a local interaction feature by computing a local interaction between a specific atom and neighboring atoms based on preprocessed data; a semi-global interaction feature extraction step S130 of extracting a semi-global interaction feature by computing an interaction between all atoms in a unit cell of a crystal based on preprocessed data; a global interaction feature extraction step S140 of extracting a global interaction feature by computing a long-range interaction in consideration of periodicity between unit cells based on preprocessed data; and a physical property prediction step S150 of integrating each feature extracted in each step and predicting a target physical property of the material based thereon.

The input data generation step S110 may transform the atomic structure data of the material to be processed by the physical property prediction model. In one embodiment of the invention, data in a standard format such as a CIF may be input. Information such as the type of each atom, three-dimensional coordinates, unit cell parameters, and the like may be extracted from the CIF data input in this step, and then three types of input data may be generated according to the features of subsequent computations steps (local, semi-global, and global).

To generate graph data for local interaction computations, a graph structure may be generated with atoms as nodes and pairs of atoms within a certain cut-off radius as edges. Each node has an initial feature vector representing element-specific features such as atomic number and electronegativity, and each edge has geometric information such as the distance between two atoms as features. This input data (graph) may be used for GNN-based local interaction computations.

For generating sequence data for a semi-global interaction computation, an input sequence listing feature vectors of all atoms in a unit cell in order and a relative distance matrix generated by calculating the Euclidean distance between all atom pairs may be generated. This relative distance matrix may be used as an attention bias in self-attention computations of a transformer to provide three-dimensional spatial information.

For generating extended graph data for global interaction computations, to model periodicity of a crystal structure, a cut-off radius larger than that of a local interaction graph may be applied, and PBCs may be applied to generate an extended graph connected to neighboring atoms beyond unit cell boundaries. This graph may be used for GNN-based global interaction computations.

The local interaction feature extraction step S120 may receive the local graph data generated in the preprocessing step as input to extract local interaction features at the atomic level. To this end, a GNN, for example, a message passing neural network (MPNN), may be used.

During the computational process, each atom (node) propagates its feature vector in the form of a ‘message’ to connected neighboring atoms, and simultaneously receives messages from the neighbors to update its own state. By repeating this message passing process across multiple layers, the final feature vector of each atom may embed intricate local chemical environment information, such as chemical bonds, distances, angles, and the like between surrounding atoms. The local interaction features extracted in this manner may provide the most fundamental information that determines material properties.

The semi-global interaction feature extraction step (S130) may use the sequence data generated in the preprocessing step to extract structural features at the entire unit cell level. A transformer architecture may be applied to comprehensively consider all interatomic interactions within the unit cell.

All atoms in the unit cell may be considered as a single sequence, and a self-attention mechanism may be applied. Self-attention calculates attention weights indicating how much one atom in the sequence is related to all other atoms. In this process, the relative distance matrix generated in the preprocessing step may be utilized as attention bias to provide three-dimensional interatomic distance information. This enables effective learning of semi-global features such as symmetry, density, and coordination environment of the entire unit cell by modeling interactions between distant atoms without direct bonding, without the cut-off radius constraint of GNN.

The global interaction feature extraction step (S140) may extract long-range interaction features between unit cells arising from the periodicity of the crystal structure. The extended graph data generated in the preprocessing step may be used for this purpose. In one embodiment, GNN may be used to extract global interaction features. When message passing computations are performed on the extended graph to which PBCs are applied, messages propagate beyond the unit cell boundaries to atoms in adjacent unit cells. Through this process, global features related to interactions between a reference unit cell and neighboring unit cells, that is, macroscopic-scale physical phenomena such as lattice strain or phonon vibrations of the entire crystal, may be extracted. Such global features provide important information for determining bulk properties of materials. In another embodiment of the invention, a transformer may be applied to extract global interaction features. For example, a special token such as a ‘class token’ may be added to the atomic sequence to train the transformer to aggregate information of the entire crystal structure into this token.

The physical property prediction step (S150) combines the local, semi-global, and global interaction feature vectors extracted in the three preceding steps into one integrated feature vector. As combination methods, concatenation of simply joining each vector or element-wise summation of adding each element of the vectors may be applied. Such integrated feature vector contains multilayer information from microscopic to macroscopic interactions of the material, and this integrated feature vector may be transmitted as input to a fully connected neural network such as an MLP to be mapped to target physical property values to be ultimately predicted (e.g., formation energy, bandgap, elastic modulus, etc.).

As shown in FIG. 5, a material physical property prediction service system 300 utilizing the physical property prediction model 200 according one an embodiment of the invention includes a user computing device 310 and a server computing system 320, and these components are communicatively connected through a service environment (e.g., an Internet site) to provide services such as physical property prediction for input molecules to the user computing device 310.

The user computing device 310 is a client terminal for requesting analysis services by uploading atomic structure data files of materials to be analyzed for physical properties, for example, CIFs, and receiving the results. The user computing device 310 may include any type of computing device capable of Internet access, for example, smartphones, tablet PCs, desktop computers, and the like.

The user computing device 310 may include a user input portion 311 (e.g., touch screen, keyboard) for receiving user input and a display 312 for outputting analysis results received from the server. Users may access the service through a web browser or dedicated application to input information about molecules to be analyzed, for example, three-dimensional molecular data in the form of prompt. The input request data may be transmitted to the server computing system 320 through a communication portion.

The server computing system 320 includes high-performance processors such as central processing units (CPU) and graphics processing units (GPU) and large-capacity memory, and it may mount and execute the physical property prediction model 200 according to one embodiment of the invention. The physical property prediction model 200 according to one embodiment may include a preprocessing module 210, a multiscale interaction computation portion, and a physical property prediction portion 250. The multiscale interaction computation portion extracts local/semi-global/global features of materials from molecular structure data input by users, and it may be configured to include a local interaction computation portion 220, a semi-global interaction computation portion 230, and a global interaction computation portion 240.

The preprocessing module 210 generates three types of data by converting atomic structure data in formats such as CIF input from users according to the characteristics of subsequent computation portions. The first input data is for computing local features and may generate a graph structure with atoms as nodes and atom pairs within specific distances as edges. The second input data is for computing semi-global features and may generate an input sequence listing feature vectors of atoms in unit cells in order and a relative interatomic distance matrix. The third input data is for computing global features and may generate an extended graph structure connected to atoms beyond unit cell boundaries considering the periodicity of unit cells. The user input data converted in this way may be respectively input to the local interaction computation portion 220, semi-global interaction computation portion 230, and global interaction computation portion 240 of the multiscale interaction computation portion to extract interaction characteristics of the molecular structure input by the user.

The local interaction computation portion 220 may apply a GNN to the first input data (graph) to extract local interaction features such as interatomic chemical bonds through message passing computations. The semi-global interaction computation portion 230 may apply a transformer architecture to the second input data (sequence) to capture interaction relationships between all atoms in a unit cell through a self-attention mechanism to extract semi-global features. At this time, three-dimensional spatial information may be reflected by using an interatomic relative distance matrix as an attention bias. The global interaction computation portion 240 may apply another GNN to third input data (extended graph) to extract global features on a macroscopic scale such as lattice strain through message passing across unit cell boundaries.

The physical property prediction portion 250 may combine the local, semi-global, and global feature vectors separately derived by the multiscale interaction computation portion into one integrated feature vector. The integrated feature vector may include all information from microscopic to macroscopic information of the material. Finally, the physical property prediction portion 250 may input the integrated feature vector into a fully connected neural network such as an MLP to predict a target physical property value requested by the user, such as formation energy, bandgap, and elastic modulus. In other words, the MLP may receive the integrated feature vector as input and calculate and output an optimal physical property value based on previously learned patterns. The finally predicted physical property value through this series of processes may be transmitted back to the user's computing device 310 through the communication portion and provided to the user.

An embodiment of the invention may be implemented as an application-specific integrated circuit (ASIC) that is manufactured to suit the specific functions of particular application fields and devices.

ASICs are also referred to as custom semiconductors. Unlike standard semiconductors that have specified standards and is applicable to any electronic product or application when certain requirements are met, ASICs are integrated circuits manufactured by semiconductor manufacturers according to specific orders for use in specific products or functions. In other words, custom semiconductors are designed and manufactured to perform only the functions necessary for specific devices or specific functions. Custom semiconductors are broadly divided into full custom ICs, in which circuits are designed and manufactured from the beginning according to user requirements based on design methods, and semi-custom ICs, in which circuits are designed and manufactured using part of standardized designs.

Custom semiconductors are mainly used in communication systems, high-performance computing systems, consumer electronics, automobiles, industrial automation, medical devices, military, aerospace industry, and the like. Recently, they have been applied to AI semiconductors that execute large-scale computations required for AI implementation with high performance and power efficiency.

ASICs are used as key components of, for example, network routers, switches, and modems in communication systems to perform data packet processing, protocol conversion, signal processing, and the like, thereby providing high throughput and low latency. In high-performance computing systems, ASICs are used as key components for high-speed processing and parallel processing, and in consumer electronics such as digital cameras, smartphones, tablets, and game consoles, ASICs provide the high-performance and low-power solutions required to perform specific functions. In the automotive industry, ASICs are used to control various electronic systems within automobiles, and in industrial automation systems, ASICs provide solutions for high-precision control and high-performance processing.

ASICs to which one embodiment of the invention is applied may include a memory in which an individual memory interface (I/F) is implemented and may include a plurality of functional blocks that request memory access. Each functional block may be a direct memory access (DMA) functional block, a processor, a video processor, a cache controller, a decompression block, or a data path block. The basic configuration of the ASICs may include transistors that amplify or switch electrical signals, logic gates that are circuits performing logical functions by combining transistors, memory cells that store data, analog circuits that are circuits processing continuous voltage or current by combining transistors, microprocessors that are pre-designed to perform specific functions, DSPs, IP cores (Intellectual Property Cores) such as graphics cores, and the like.

The ASICs may also include an individual memory I/F that interfaces with individual memory and an embedded memory I/F that interfaces with embedded memory, and the individual memory I/F may be connected to each functional block to receive memory access signals (e.g., control signals, address signals, and data signals) and generate signals for controlling the individual memory based on these input signals. The embedded memory I/F may be connected to each functional block to receive memory access signals (e.g., control signals, address signals, data signals) and generate modified memory access signals for controlling the embedded memory based on these input signals. The individual memory I/F and the embedded memory I/F are designed within a memory control block of the ASIC to provide a memory control structure that may be flexibly applied to both individual memory and embedded memory.

In addition, an ASIC for an ANN includes a plurality of neurons arranged in an array and a plurality of synaptic circuits, and each neuron may include a register, a microprocessor, and at least one input, and each synaptic circuit may include a memory for storing synaptic weights. Here, each neuron of the ASIC may be connected to at least one other neuron through one of the plurality of synaptic circuits.

Although the inventive concepts have been described above as generally being implementable by a computing device, those skilled in the art will appreciate that the present disclosure may be implemented in combination with computer-executable instructions and/or other program modules executable on one or more computers and/or as a combination of hardware and software.

Those skilled in the art will understand that information and signals may be represented using any of a variety of different technologies and techniques. For example, data, instructions, commands, information, signals, bits, symbols, and chips that may be referenced in the above description may be represented by voltages, currents, electromagnetic waves, magnetic fields or particles, optical fields or particles, or any combination thereof.

Those skilled in the art will appreciate that the various exemplary logical blocks, modules, processors, means, circuits, and algorithm steps described in connection with the embodiments disclosed herein may be implemented by electronic hardware, various forms of program or design code (referred to herein as software for convenience), or combinations of both. To clearly describe this interchangeability of hardware and software, various illustrative components, blocks, modules, circuits, and steps have been described above generally in terms of their functionality. Whether such functionality is implemented as hardware or software depends upon the particular application and design constraints imposed on the overall system. Those skilled in the art may implement the described functionality in varying ways for each particular application, but such implementation decisions should not be interpreted as departing from the scope of the present disclosure.

The various embodiments presented herein may be implemented as a method, apparatus, or article of manufacture using standard programming and/or engineering techniques. The term “article of manufacture” includes a computer program, carrier, or media accessible from any computer-readable storage device. For example, computer-readable storage media include magnetic storage devices (e.g., hard disks, floppy disks, magnetic strips, etc.), optical disks (e.g., CDs, DVDs, etc.), smart cards, and flash memory devices (e.g., EEPROMs, cards, sticks, key drives, etc.), but are not limited thereto. In addition, the various storage media presented herein include one or more devices and/or other machine-readable media for storing information.

It should be understood that the specific order or hierarchy of steps in the processes presented is an example of exemplary approaches. It should be understood that, based on design priorities, the specific order or hierarchy of steps in the processes may be rearranged within the scope of the present disclosure. The accompanying method claims present elements of various steps in a sample order, but are not meant to be limited to the specific order or hierarchy presented.

According to embodiments of the invention, by separating and learning interatomic interactions in a material into three physical scales of local, semi-global, and global scales, multiscale physical phenomena that are difficult to capture with a single model can be comprehensively considered, thereby enabling more accurate and reliable physical property prediction. Furthermore, by individually applying architectures that can best capture features of each scale and integrating the results, the accuracy of material physical property prediction can be dramatically improved compared to existing methods, thereby contributing to dramatically reducing time and cost in new material development processes.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A system, comprising:
at least one processor; and
at least one memory storing instructions, information, or an artificial intelligence model executed by the at least one processor,
wherein the instructions, information, or artificial intelligence model executed by the at least one processor includes:
a preprocessing module configured to receive atomic structure data of the material and generate first input data for a local interaction computation, second input data for a semi-global interaction computation, and third input data for a global interaction computation;
a local interaction computation portion configured to compute local interactions between a specific atom and neighboring atoms around the specific atom based on the first input data to extract a local interaction feature;
a semi-global interaction computation portion configured to compute interactions between all atoms in a unit cell of a crystal based on the second input data to extract a semi-global interaction feature; and
a global interaction computation portion configured to compute long-range interactions in consideration of periodicity between unit cells based on the third input data to extract a global interaction feature.

2. The system according to claim 1, wherein the local interaction computation portion includes a graph neural network, and the first input data that is input to the graph neural network has a graph structure in which atoms of the material are nodes and atom pairs located within a specific cut-off radius of the material are edges.

3. The system according to claim 1, wherein the semi-global interaction computation portion includes a transformer, and the second input data that is input to the transformer includes an input sequence listing feature vectors of atoms in the unit cell in order.

4. The system according to claim 3, wherein the semi-global interaction computation portion extracts the semi-global interaction feature by using a relative distance matrix between all atom pairs in the unit cell as an attention bias of a self-attention computation.

5. The system according to claim 1, wherein the global interaction computation portion includes another graph neural network, and the third input data that is input to the other graph neural network is an extended graph structure connected to neighboring atoms beyond a boundary of the unit cell.

6. The system according to claim 1, further comprising a physical property prediction portion,
wherein the physical property prediction portion is configured to generate an integrated feature vector by integrating respective feature vectors of the local interaction feature, the semi-global interaction feature, and the global interaction feature.

7. The system according to claim 6, wherein the physical property predicting portion predicts a target physical property by inputting the integrated feature vector to a multilayer perceptron.

8. The system according to claim 1, wherein the preprocessing module obtains the atomic structure data by receiving a crystallographic information file (CIF).

9. A method, which is a computerized method, comprising:
generating input data including first input data for local interaction computation, second input data for semi-global interaction computation, and third input data for global interaction computation by receiving a crystallographic information file (CIF) as atomic structure data of the material;
extracting a local interaction feature by computing local interactions between a specific atom and surrounding neighboring atoms thereof based on the first input data;
extracting a semi-global interaction feature by computing, based on the second input data, interactions between all atoms in a unit cell of a crystal;
extracting a global interaction feature by computing, based on the third input data, long-range interactions considering periodicity between unit cells; and
predicting a target property of the material by integrating the local interaction feature, the semi-global interaction feature, and the global interaction feature.

10. The method of claim 9, wherein the generating input data generates, through a preprocessing module, the first input data having a graph structure in which atoms of the material are nodes and atom pairs located within a specific cut-off radius of the material are edges.

11. The method of claim 10, wherein the extracting a local interaction feature extracts the local interaction feature through a message passing computation of a graph neural network on the first input data.

12. The method of claim 9, wherein the generating input data generates the second input data including an input sequence listing feature vectors of atoms in the unit cell in order and a relative distance matrix obtained by calculating distances between all atom pairs.

13. The method of claim 12, wherein the extracting a semi-global interaction feature extracts the semi-global interaction feature by applying a self-attention mechanism of a transformer architecture to the second input data and uses the relative distance matrix as an attention bias.

14. The method of claim 9, wherein the generating input data generates the third input data of an extended graph structure connected to neighboring atoms beyond a boundary of the unit cell.

15. The method of claim 14, wherein the extracting a global interaction feature extracts the global interaction feature including interactions between unit cells by applying a graph neural network to the extended graph structure of the third input data.

16. The method of claim 9, wherein the predicting a target property of the material predicts the target property of the material by combining vectors representing each of the local interaction feature, the semi-global interaction feature, and the global interaction feature to generate an integrated feature vector and passing the integrated feature vector through a fully connected neural network.

17. A service system, comprising:
a user computing device configured to transmit an analysis request for atomic structure data of the material and receiving a predicted physical property value; and
a server computing system communicatively connected with the user computing device,
wherein the server computing system includes:
a preprocessing module configured to receive the atomic structure data as input and generating first input data for local interaction computation, second input data for semi-global interaction computation, and third input data for global interaction computation;
a multiscale interaction computation portion configured to extract a local interaction feature based on the first input data, a semi-global interaction feature based on the second input data, and a global interaction feature based on the third input data; and
a physical property prediction portion configured to predict a target physical property of the material by integrating the extracted respective interaction features.

18. The system of claim 17, wherein the multiscale interaction computation portion includes:
a local interaction computation portion configured to extract the local interaction feature by applying message passing computation of a graph neural network to the first input data;
a semi-global interaction computation portion configured to extract the semi-global interaction feature by applying self-attention of a transformer to the second input data; and
a global interaction computation portion configured to extract the global interaction feature by applying message passing computation of a graph neural network to the third input data.

19. The system of claim 17, wherein the physical property prediction portion predicts the target physical property by combining respective feature vectors representing the local interaction feature, the semi-global interaction feature, and the global interaction feature to generate an integrated feature vector and inputting the integrated feature vector into a multilayer perceptron.

20. An application-specific integrated circuit, configured as a functional block, including:
a memory configured to store information, instructions, and an artificial intelligence model; and
at least one processor configured to request access to the memory,
wherein the memory stores an artificial intelligence model, instructions, or information receiving a crystallographic information file as atomic structure data of the material and generating first input data including first input data for local interaction computation, second input data for semi-global interaction computation, and third input data for global interaction computation; extracting a local interaction feature by computing local interactions between a specific atom and surrounding neighboring atoms thereof based on the first input data; extracting a semi-global interaction feature by computing interactions between all atoms within a unit cell of a crystal based on the second input data; extracting a global interaction feature by computing long-range interactions considering periodicity between unit cells based on the third input data; and predicting a target physical property of the material by integrating the local interaction feature, the semi-global interaction feature, and the global interaction feature.
